(19) United States
(12) Patent Application Publication
(10) Pub. No.: US 2006/0006089 A1
(43) Pub. Date: Jan. 12, 2006

(54) TOILET PAPER ROLL SCENTING METHOD

(76) Inventor: Ahmad R. Arash, West Newbury, MA (US)

Correspondence Address: MESMER & DELEAULT, PLLC, 1 NEW HAMPSHIRE AVE., SUITE 125, PORTSMOUTH, NH, 03801, US

(21) Appl. No.: 10/711,977

(22) Filed: Oct. 18, 2004

Related U.S. Application Data
(60) Provisional application No. 60/521,546, filed on May 19, 2004.

(51) Int. Cl. B65D 85/676; A61L 9/12; A47K 10/16; B65B 29/00

(52) U.S. Cl. 206/410; 239/52; 53/430; 242/160.1

(57) ABSTRACT

A method for scenting toilet paper that does not require adding special apparatuses to the toilet paper dispensing means. The method includes the steps of providing a tubular toilet paper roll made of an absorbent material, applying a liquid scent to the toilet paper roll, attaching toilet paper onto the roll to form a toilet paper roll, and wrapping the toilet paper roll substantially air tight with a material impervious to the scent, thereby scenting the toilet paper roll.

### BACKGROUND

1. Field of the Invention

The invention relates to a method for diffusing a scent into a toilet paper roll using liquid scent applied to an absorbent roll.

2. Description of the Related Art

Past methods of scenting toilet paper have always required the addition of a special apparatus to the toilet paper dispensing means. Examples include three of the cage-type toilet paper rollers that have been patented, but are not admitted to being prior art by their mention in this Background section. The three examples are U.S. Pat. No. 3,017,117 that issued to Klingler; U.S. Pat. No. 4,759,51 that issued to Singer; and U.S. Pat. No. 6,688,551 that issued to He et al. In each of these three examples, a roller is provided separately from the toilet paper. A user replaces an original roller with the special cage-type roller. The user must load the roller with a scent source, and must periodically replace or reload the scent source. The scent is emitted through the gaps in the cage, or through holes in the roller body. Because of the gap between the outside diameter of the roller and the inside diameter of a toilet paper roll, the release of scent is likely to be constant over time. There is no greater gap through which to release the scent when the roller is in use versus when the roller is not in use. Therefore, scent is released whether it is needed or not.

Another solution that has been tried is disclosed in U.S. Pat. No. 6,575,383 that issued to Dobler et al., and is also not admitted to being prior art by its mention in this Background section. Dobler discloses disposing a pre-scented folded card insert that is designed to fit within an existing toilet paper holder. In use, the card is outside the roller, but inside the roll. Since the card is folded instead of rolled, it is unlikely that the toilet paper will unwind smoothly off the roll. Another embodiment of this invention is to place the card inside the roller. However, it would not be effective unless it is a cage-type roller discussed above. In either embodiment, scenting the toilet paper still requires adding a separate apparatus to the system, in this case the card. Like with the above references, there is no greater gap through which to release the scent when the roller is in use versus when the roller is not in use. Therefore, scent is released whether it is needed or not.

Furthermore, in each of the references, getting the toilet paper scented requires much more effort and expense than using unscented toilet paper. What is needed, therefore, is a method for scenting toilet paper that does not require adding special apparatuses to the toilet paper dispensing means, and does not require any greater effort than non-scented toilet paper to use.

### SUMMARY

A method for scenting toilet paper that does not require adding special apparatuses to the toilet paper dispensing means, and does not require any greater effort than non-scented toilet paper to use, comprises the steps of providing a tubular toilet paper roll made of an absorbent material, applying a liquid scent to the toilet paper roll, disposing toilet paper onto the roll to form a toilet paper roll, and wrapping the toilet paper roll substantially air tight with a material impervious to the scent, thereby scenting the toilet paper roll. These and other features, aspects, and advantages of the present invention will become better understood with regard to the following description, claims, and accompanying drawings.

### DRAWINGS

FIG. 1 is a perspective view of a wrapped toilet paper roll scented according to the method of the present invention.

FIG. 2 is a cross section view of the wrapped toilet paper roll scented according to the method of the present invention.

FIG. 3 is a flow chart describing the preferred method of the present invention.

### DESCRIPTION

A method for scenting toilet paper that does not require adding special apparatuses to the toilet paper dispensing means, and does not require any greater effort than non-scented toilet paper to use, comprises the steps of providing a tubular toilet paper roll made of an absorbent material, applying a liquid scent to the toilet paper roll, disposing toilet paper onto the roll to form a toilet paper roll, and wrapping the toilet paper roll substantially air tight with a material impervious to the scent, thereby scenting the toilet paper roll.

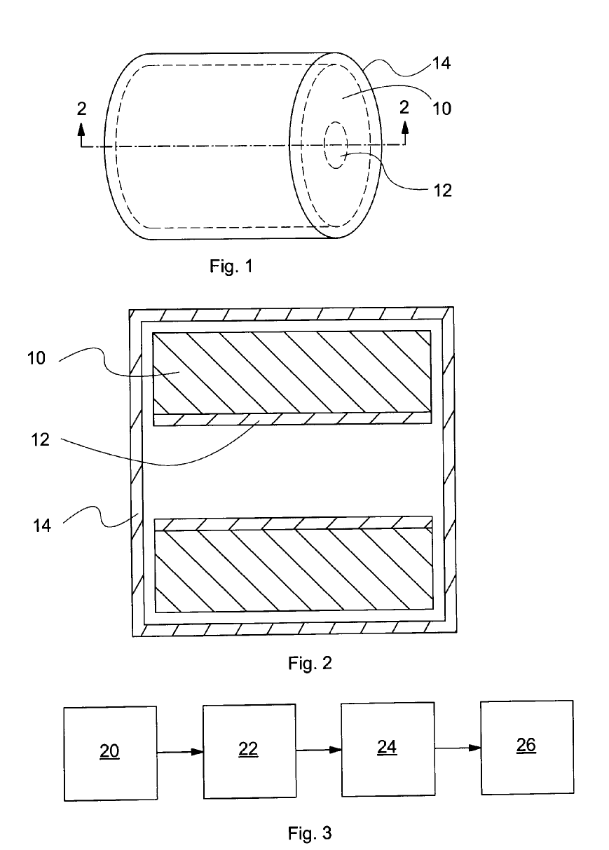

Turning to FIGS. 1, 2, and 3, the method is preferably started at the toilet paper manufacturing process. First, a tubular toilet paper roll 12 is provided 20. The roll 12 must be made of some material that is capable of absorbing a liquid scent. An example is cardboard, but it is not limited to this material.

Then, a liquid scent is applied 22 to the roll 12. The application can be by spraying, rolling, dripping, dipping, diffusing, or other method known to those having skill in the art.

Then, toilet paper 10 can be disposed 24 onto the roll 12 form a toilet paper roll. The typical disposing step is done by applying an adhesive to the roll or to the paper, and then rolling the paper onto the roll. Other methods can be used. It can also be appreciated that the steps of applying the scent 22 and disposing the paper onto the roll 24 can be reversed.

Finally, the toilet paper roll is wrapped 26 with a material impervious to the scent. The roll is wrapped substantially air tight to keep the scent inside. After this step, and before the paper is used by the consumer, the scent diffuses naturally through the toilet paper until it is uniformly scented.

In use, the user unwraps the scented toilet paper roll, and places it in an ordinary toilet paper dispenser in a bathroom. The outside layers of the toilet paper are exposed to the air, where those layers give up its scent to freshen the bathroom air. After they give up their scent, little further scent can be released by the outside layers.

Then, when one is using the bathroom, the user unrolls at least a portion of the toilet paper, thereby exposing fresh scented toilet paper to the bathroom air. In this manner, the scent is released precisely when it is needed. The toilet paper roll does not have to be supplied with a separate scent holder, or any other apparatus, to operate. This saves a great deal of money and reduces waste.

While there have been described what are at present considered to be the preferred embodiments of this invention, it will be obvious to those skilled in the art that various changes and modifications may be made therein without departing from the invention, and it is, therefore, aimed to cover all such changes and modifications as fall within the true spirit and scope of the invention.

What is claimed is:

1. A method for scenting a toilet paper roll comprising the steps of:
providing a tubular toilet paper roll made of an absorbent material,
applying a liquid scent to the toilet paper roll,
disposing toilet paper onto the roll to form a toilet paper roll, and
wrapping the toilet paper roll substantially air tight with a material impervious to the scent, thereby scenting the toilet paper roll.

2. A method for diffusing a pleasing scent to a bathroom when it is needed comprising the steps of:
providing a roll of toilet paper having a scented roll, and
unrolling at least a portion of the toilet paper at the time of use to expose the toilet paper to the bathroom air, thereby freshening the bathroom air when it is needed.

3. The method of claim 2, the step of providing a roll of toilet paper having a scented roll comprising the steps of:
providing a tubular toilet paper roll made of an absorbent material,
applying a liquid scent to the toilet paper roll,
disposing toilet paper onto the roll to form a toilet paper roll, and
wrapping the toilet paper roll substantially air tight with a material impervious to the scent, thereby scenting the toilet paper roll.

4. The product made by the process of claim 3.

5. A scented toilet paper roll comprising:
a tubular roll made of a material capable of absorbing a scent;
a liquid scent capable of being absorbed by the tubular roll;
toilet paper disposed on the tubular roll; and
an air tight wrapping wrapped around the toilet paper, roll, and scent, whereby the scent absorbed by the roll is diffused through the toilet paper while wrapped by the wrapping.
